(19) United States
(12) Patent Application Publication
(10) Pub. No.: US 2022/0401395 A1
(43) Pub. Date: Dec. 22, 2022

(54) MUCOADHESIVE COMPOSITIONS AND METHOD OF USE THEREOF

(71) Applicant: ISP INVESTMENTS LLC, Wilmington, DE (US)

(72) Inventors: Sounak SARKAR, Hillsborough, NJ (US); Shafiq Sahar WAHIDI, Pomona, NY (US); Petros GERESELASSIE, Whitehouse Station, NJ (US); Philip John OTHS, Mendham, NJ (US); Hani M. FARES, Somerset, NJ (US)

(73) Assignee: ISP INVESTMENTS LLC, Wilmington, DE (US)

(21) Appl. No.: 17/764,511

(22) PCT Filed: Aug. 24, 2020
(86) PCT No.: PCT/US20/47697

§ 371 Date: Mar. 28, 2022

Related U.S. Application Data
(60) Provisional application No. 62/891,597, filed on Aug. 26, 2019.

(51) Int. Cl. A61K 31/194; A61K 31/79; A61K 31/717; A61K 45/06

(52) U.S. Cl. CPC A61K 31/194; A61K 31/79; A61K 31/717; A61K 45/06

(57) ABSTRACT

A mucoadhesive composition including denture adhesive composition comprising: (i) 10 to about 75 wt. % of a maleic acid or anhydride copolymer; (ii) 10 to about 50 wt. % of a cellulose ether; (iii) 0.1 to 10 wt. % of a crosslinked polyvinyl pyrrolidone that is swellable but not soluble in water; and (iv) 30 to 70 wt. % of an orally acceptable carrier based on the total weight of the composition. Also disclosed is a method of use thereof and a process lot preparing the same.

### FIELD OF THE INVENTION

I The present application relates to a mucoadhesive composition and More particularly, to a mucoadhesive composition including denture adhesive composition comprising (i) maleic acid or maleic anhydride copolymer or salts of maleic acid or maleic anhydride copolymer; (ii) a cellulose ether: (iii) a crosslinked polyvinyl pyrrolidone polymer that is swellable but insoluble in water; and (iv) a hydrophobic oil or hydrophilic non-oil components as a carrier.

### BACKGROUND OF THE INVENTION

Mucoadhesive compositions, including denture adhesive formulations, available generally in the form of creams, powders or extruded dry wafers/films, are used to provide secure hold of artificial dentures in mouth for users of partial or full dentures or deliver therapeutic actives to desired areas inside buccal cavity by topical applications. Typically, a mucoadhesive compositions including denture adhesive cream consists of water soluble or water swellable adhesive polymers suspended in an orally acceptable carrier matrix generally composed of different proportions of long chain hydrocarbons or long chain fatty acids like mineral oils, vegetable oils, petrolatum, etc. During use, activation of the adhesive components in mucoadhesive composition, particularly denture adhesive cream occurs on contact with saliva, resulting in hydration, swelling and subsequent buildup of adhesive and cohesive hold of the denture adhesive formulation applied between the gum and the denture. Duration and strength of adhesive hold provided by a denture adhesive formulation is the primary quantitative performance parameter of the denture adhesive. Other semi-quantitative or qualitative parameters that are used to define overall performance characteristics of a denture adhesive formulation might include film thickness and cushioning effect provided by the denture adhesive between gum and denture, preventing food particles from entering interstitial space between gum and denture by providing superior gap sealing and loss of denture adhesive material from between gum and denture due to oozing during its time of use. Since common mucoadhesive composition including denture adhesive creams are essentially suspensions or dispersions of solid active ingredients in a semi-solid or liquid matrix composed of various kinds of oils, loss of shelf life stability of the formulation due to syneresis and separation of the carrier matrix and solid active ingredients might also adversely affect adhesion performance.

Thus, an ideal mucoadhesive composition, particularly denture adhesive cream should have suitable shelf-life stability over a sustained period of time, provide sufficiently high instant dry tack and prolonged adhesive hold during use, provide adequate cushioning between gum and denture to ensure comfort of use, be able to seal gaps between denture and gum to prevent food particles from entering the gap, have reduced uncomfortable mouth feel due to oozing out of partially hydrated denture adhesive from between gum and denture and easy cleanability after use.

U.S. Pat. No. 3,440,065A describes the use of sodium salt of carboxymethyl cellulose (Sodium CMC) as an active adhesive ingredient in a denture adhesive formulation.

U.S. Pat. No. 4,514,528A describes the use of partial sodium/calcium mixed salt of lower alkyl vinyl ether-maleic anhydride copolymers, more specifically methyl vinyl ether-maleic anhydride copolymers, as an adhesive active in denture adhesive formulations.

U.S. Pat. No, 4,569,955A describes a combination of sodium CMC and Ca/Na salts of copolymers of lower alkyl vinyl ether-maleic anhydride, specifically, methyl vinyl ether-maleic anhydride in denture adhesive formulations. Since then, a class of synthetic copolymers of lower alkyl vinyl ether, most prevalently methyl vinyl ether, and maleic anhydride, metal salts of these polymers or metal crosslinked forms of these polymers or a mixture of these along with different types of water-soluble cellulosic materials, most prevalently, but not restricted to Sodium CMC, has been used in oil-based denture adhesive formulations.

DE3228335A1 discloses the use of polyvinylpyrrolidone (PVP) as an adhesive active for denture adhesive formulations.

EP2620136B1 and U.S. Pat. No. 9,408,780B2 describe the use of PVP in denture adhesive formulations for the purpose of improvement of initial dry tack for oil-based denture adhesive formulations, as an adhesive active for oil-free denture adhesive formulations, as a formulation and stabilization aid and as a hydration promoter for oil-based denture adhesive formulations.

Although hydration of the adhesive actives in contact with saliva during use is essential to activate and build adhesive characteristics of mucoadhesive composition, particularly denture adhesives, the rate and extent of hydration of the adhesive actives is crucial to the overall performance of the mucoadhesive composition, particularly denture adhesive formulation. Adhesive actives like Ca/Na salts of copolymers of lower alkyl vinyl ether-maleic anhydride, specifically methyl vinyl ether-maleic anhydride, water soluble cellulosics like CMC and lactamic polymers like PVP hydrate and eventually, over the course of time of use, dissolve in saliva. Loss of dissolved adhesive actives from between gum and denture appliances in the form of “ooze” negatively impacts adhesive hold, cushioning, food seal and causes uncomfortable mouthfeel.

The present application describes qualitative and quantitative improvements in the overall performance characteristics of mucoadhesive composition, particularly denture adhesive compositions achieved through incorporation of hydrophobically modified polymeric adhesive actives in oil-based and oil-free mucoadhesive composition including denture adhesive formulations. Hydrophobic modifications ranging from light substitution of short to long chain hydrophobes on polymeric backbone to light or heavy intra and/or interchain crosslinking of polymeric backbone with hydrophobic crosslinkers render common water soluble polymeric adhesive actives sparingly water soluble, water dispersible or totally water insoluble. At the same time, due to their analogous chemical structures and side groups similar to common polymeric adhesive actives, these hydrophobically modified polymers retain their inherent adhesive functionality, in addition to performing as cohesion builders by creating water insoluble, but water swellable, interpenetrating networks in the hydrated mucoadhesive composition including denture adhesive hydrogel. Incorporation of hydrophobically modified adhesive polymers either as additives or as total replacement of one or more adhesive active in standard oil-based and oil-free mucoadhesive composition including denture adhesive formulations showed improved denture adhesive performance in terms of strength and duration of adhesive hold, film thickness and cushioning effects, ooze control, shell-life storage stability and easy cleanability in comparison to benchmark formulation.

### SUMMARY OF THE INVENTION

The primary aspect of the present application is to provide an improved mucoadhesive composition comprising: (i) about 10 to about 75 wt. % of a maleic acid or maleic anhydride copolymer or salts of a maleic acid or maleic anhydride copolymer; (ii) about 10 to about 50 wt. % of at least one cellulose ether; (iii) about 0.1 to about 10 wt. % of a crosslinked polyvinyl pyrrolidone polymer which is swellable but not soluble in water; and (iv) about 30 to about 70 wt. % of an orally acceptable carrier.

One aspect of the present application provides an mucoadhesive oral composition comprising: (i) about 10 to about 75 wt. % of a maleic acid or maleic anhydride copolymer or salts of a maleic acid or maleic anhydride copolymer; (ii) about 10 to about 50 wt. % of at least one cellulose ether; (iii) about 0.1 to about 10 wt. % of a crosslinked polyvinyl pyrrolidone polymer which is swellable but not soluble in water; (iv) about 30 to about 70 wt. % of an orally acceptable carrier; and (v) about 0.01 to about 20 wt. % of an active ingredient selected from the group consisting of an antibacterial, an anti-inflammatory, a pain reliving agent, an antioxidant, an enzyme, a flavor, a cooling agent, a sweetener and mixtures thereof and further wherein the composition is in the form of either as a gel or a liquid.

Another aspect of the present application provides an denture composition comprising: (i) about 10 to about 75 wt. % of a maleic acid or maleic anhydride copolymer or salts of a maleic acid or maleic anhydride copolymer; (ii) about 10 to about 50 wt. % of at least one cellulose ether: (iii) about 0.1 to about 10 wt. % of a crosslinked polyvinyl pyrrolidone polymer which is swellable but not soluble in water; (iv) about 30 to about 70 wt.% of an orally acceptable carrier; and (v) about 0.01 to about 20 wt. % of an active ingredient selected from the group consisting of an antibacterial, an anti-inflammatory, a pain reliving agent, an antioxidant, an enzyme, a flavor, a cooling agent, a sweetener and mixtures thereof and further wherein the composition is in the form of either as a gel or a liquid.

One aspect of the present application provides a mucoadhesive composition including denture adhesive composition, wherein the maleic acid copolymer or maleic anhydride copolymer comprises maleic acid or maleic anhydride and a C₃—C₄ alkyl vinyl ether having a predetermined weight average molecular weight of about 500,000 to 3,000,000 suitable for denture adhesives made by copolymerizing about 50 mole percent of maleic anhydride, about 50 mole percent of a C₁—C₄ alkyl vinyl ether, in the presence of a free radical initiator, at about 50° to 150° C., in a solvent or solvent free process to produce a uniform, fine powder having substantially no residual maleic anhydride.

Another aspect of the present application provides a mucoadhesive composition including denture adhesive composition, wherein the methyl vinyl ether-maleic acid (MVE-MA) copolymer or methyl vinyl ether-maleic anhydride (MVE-MA) copolymer has a number average molecular weight of between about 50,000 and about 500,000 and wherein from about 50 to about 100 wt. % of the carboxyl units in the polymers are converted to a mixture of metal salts, for which the metals can be selected from the group consisting of calcium, sodium, strontium, zinc, magnesium, iron, boron, aluminum, potassium, vanadium, chromium, manganese, nickel, copper, yttrium, titanium, and mixtures thereof.

Yet another aspect of the present application discloses a mucoadhesive composition including denture adhesive composition, wherein the salts of copolymer of alkyl vinyl ether-maleic acid or salts of alkyl vinyl ether-maleic anhydride have a specific viscosity of from 2.5 to 5.0 when measured as a 1% w/v solution in methyl ethyl ketone (MEK) solution at 25° C.

In another aspect, the present application discloses a mucoadhesive composition including denture adhesive composition comprising: (i) about 15 to about 70 wt. % maleic acid copolymers selected from the group consisting of methyl vinyl ether-maleic acid (MVE-MA) copolymer or methyl vinyl ether-maleic anhydride (MVE-MA) copolymer or salts thereof; about 10 to about 50 wt. % carboxymethylcellulose (CMC); about 0,1 to about 10 wt. % of a water insoluble crosslinked polyvinyl pyrrolidone polymer; and from about 10 to about 60 wt. % of an orally acceptable carrier.

One other aspect of the present application discloses a method of adhering a denture having a biocontact surface to a gum or a roof of a mouth, the method comprising treating the biocontact surface of the denture with a mucoadhesive composition, particularly denture adhesive composition comprising: (i) about 15 to about 70 wt. % maleic acid copolymer selected from the group consisting of a methyl vinyl ether-maleic acid (MVE-MA) copolymer or methyl vinyl ether-maleic anhydride (MVE-MA) copolymer or salt thereof; (ii) about 1.0 to about 50 wt. % of carboxymethylcellulose; (iii) about 0.1 to about 10 wt. % of a water insoluble crosslinked polyvinyl pyrrolidone polymer; (iv) about 10 to about 60 wt. % of an orally acceptable carrier; and (y) about 0.01 to about 20 wt. % of an active ingredient selected from the group consisting of an antibacterial, an anti-inflammatory, a pain reliving agent, an antioxidant, an enzyme, a flavor, a cooling agent, a sweetener and mixtures thereof and further wherein the composition is either a gel or a liquid.

### BRIEF DESCRIPTION OF THE FIGURES

Further embodiments of the present application can be understood with the appended figures.

FIGS. 1-4 represent overlay of average adhesion profiles of sample oil-based denture adhesives Comparative Example E1, E12-16.

FIG. 5 shows the Total Adhesion for each representative denture adhesive formulation.

FIG. 6 represents overlay of average adhesion profiles of sample oil-free denture adhesives OF1 and OF2.

FIG. 7 shows the Total Adhesion for each representative oil-free denture adhesive formulation

FIG. 8 shows stability of selected oil-based denture adhesive formulations after 12 weeks at 45° C.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated., shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The term “mucoadhesive” refers to an adhesive that sticks to or adheres to the skin or mucus or mucosal cell or dental surfaces.

As used herein, “denture” refers to either partial or full upper or lower denture, or all of the above. Desirably, composition should function as an effective means for insulating, cushioning and securely positioning the denture. The composition should retain its characteristics and properties in the powder and cream forms during storage under various climatic conditions, such as temperature and humidity; be readily and easily capable of application to the denture surface; not be irritating or uncomfortable to the user; be safe and nontoxic; have no disagreeable odor or color, have no unpalatable taste; provide antiseptic and germicidal properties for preventing or inhibiting the growth of organisms ordinarily found in the mouth; and function as a deodorant or agent for prevention of putrefaction: or malodorous decomposition of foods of secretions lodging beneath or adjacent to the denture.

As used herein, “alkyl vinyl ether copolymer” refers to a predetermined weight average molecular weight of from about 500,000 to 3,000, 000 suitable for denture adhesives made by: copolymerizing about 50 mole percent of maleic anhydride, about 50 mole percent of a C₁—C₄ alkyl vinyl ether, in the presence of a free radical initiator, at about 50° to 150° C. with solvent or solvent-free process to produce as a uniform, fine powder having substantially no residual maleic anhydride.

The alkyl vinyl ether maleic anhydride copolymers are obtained by co-polymerizing an alkyl vinyl ether monomer, such as methyl vinyl ether, ethyl vinyl ether, divinyl ether, propyl vinyl ether and isobutyl vinyl ether, with maleic anhydride to yield the corresponding alkyl vinyl ether-maleic anhydride copolymer which is readily hydrolysable to the acid copolymer. Both anhydride and acid forms are also available from commercial suppliers. For example, Ashland LLC provides both the polymeric free acid form and the corresponding anhydride form under its “GANTREZ” trademark as the “GANTREZ S Series” and “GANTREZ AN Series”, respectively.

As used herein, “cellulose ether” refers to a cellulose derivative obtained by etherifying a hydroxyl group of cellulose using an etherifying agent. Useful cellulose ethers can be selected from the group consisting of methylcellulose, hydroxyethylcellulose, hydroxypropylcellulose, hydroxypropylmethylcellulose, hydrophobically modified hydroxyalkyl cellulose, sodium carboxymethylcellulose and mixtures thereof.

The mucoadhesive composition including denture adhesive composition of the present application comprise metal salts of an alkyl vinyl ether-maleic acid/maleic anhydride copolymer, wherein, the metals are selected from the group consisting of calcium, sodium, strontium, zinc, magnesium, iron, boron, aluminum, potassium, vanadium, chromium, manganese, nickel, copper, yttrium, titanium, and mixtures thereof.

The salt form of the methyl vinyl ether-maleic acid/maleic anhydride copolymers can be prepared by the interaction of the methyl vinyl ether-maleic acid/maleic anhydride copolymer or terpolymer with at least one inorganic salt aft metal, such as calcium, sodium, strontium, zinc, magnesium, iron, boron, aluminum, potassium, vanadium, chromium, manganese, nickel, copper, yurium, titanium, and mixtures thereof. In an embodiment, sodium hydroxide or calcium hydroxide are utilized for the purposes of this application.

As used herein “crosslinked” refers to a composition containing intra-molecular and/or inter-molecular crosslinks, whether arising through covalent or non-covalent bonding. “Non-covalent” bonding includes both hydrogen bonding and electrostatic (ionic) bonding.

According to one embodiment of the present application, there is employed a strongly swellable, moderately crosslinked PVP (polyvinylpyrrolidone) polymer available under the tradename FlexiThix™ from Ashland LLC obtained directly as a fine powder by precipitation polymerization of vinyl pyrrolidone in the presence of a predetermined amount of a multifunctional crosslinking agent and a free radical initiator in an organic solvent. The crosslinked PVP has a Brookfield viscosity of at least about 500 to about 50,000 cps in 4% aqueous solution. The preferred viscosity ranges of the crosslinked PVP in the present application can be varied from about 500 to about 50,000 cps or from about 800 to about 20,000 cps or from about 1000 to about 10,000 cps. The Brookfield viscosity can be measured at 2.5, 5, 10, 12, 20, 30, or 50 RPM and at 25° C.

The crosslinked PVP polymer useful in the practice of the present application can be prepared according to granted U.S. Pat. No. 5,073,614, and U.S. Pat. No. 5,130,388 assigned to ISP Investments Inc. The teachings of these references are advantageously employed for the purposes of the present application. Further, the references are incorporated herein by reference in their entirety.

An element of the thickening additive composition according to the present application is a thickening agent comprising a strongly swellable, lightly to moderately crosslinked polyvinylpyrrolidone as described in commonly owned U.S. Pat. Nos. 5,312,619 and 5,139,770, the contents of which are hereby incorporated by reference in their entirety. The term “strongly swellable, lightly to moderately crosslinked PVP”, unless otherwise noted, specifically refers to a polymer essentially consisting of lightly-to moderately-crosslinked poly(N-vinyl-2-pyrrolidone) having at least one of the following non-limiting characteristics: (1) an aqueous swelling parameter defined by its gel volume from about 15 mL/g to about 300 mL/g, more particularly from about 15 mL/g to about 250 mL/g, and in other cases from about 15 mL/g to about 150 mL/g, or (2) a Brookfield viscosity of (measured at 5% crosslinked PVP in a liquid carrier comprising water at 25° C.) of at least 2,000 cP, more particularly preferably of at least about 5,000 cP, and in certain cases of at least about 10,000 cP. Reference for these parameter ranges is provided in U.S. Pat. No. 5,073,614 (incorporated herein by reference) and in Shih, J. S., et al. (1995). Synthesis methods for the crosslinked PVP are disclosed in a number of references, including U.S. Pat. Nos. 5,073,614; 5,654,385; and 6,177,068, the contents of which are hereby incorporated by reference. It is appreciated by a polymer scientist skilled in the art that various methods of synthesis are possible, provided that the produced polymer achieves at least one of the above defined parameters.

It is contemplated to employ any other possible polymerization methods known in the art for a skilled artisan, which can provide such strongly swellable, moderately crosslinked. PVP as a fine white powder. Such polymerization methods can include, but are not limited to, precipitation polymerization, inverse emulsion polymerization, gel polymerization, dispersion polymerization, solution polymerization, emulsion polymerization , bulk polymerization, suspension polymerization, Liquid dispersion polymerization (LDP) and ionic polymerization.

Other preferred polymerization techniques employed to prepare the polymer of the present application are disclosed in (1) “Principles of Polymerization” 4^{th} edition, 2004, Wiley by George Odian and (2) WO2012061147A1 assigned to ISP Investments Inc., the disclosures of which are incorporated herein by reference in their entirety. Further, the polymerization of the polymer useful in the practice of the present application can optionally require suitable catalysts or initiators, stabilizers, salts, pH adjusting agents, co-dispersants, thickeners, solvents, acidic agents, basic agents, and/or photo initiators depending on type of polymerization technique being employed. One skilled in the art can easily derive such information from the relevant literature known in the art or from “Principles of Polymerization” 4^{th} edition, 2004, Wiley by George Odian, which is incorporated herein by reference in its entirety.

In an embodiment, the polymer is poly-N-vinyl-poly-2-pyrrolidone. The poly-N-vinyl-poly-2-pyrrolidone is also commonly known as polyvinylpyrrolidone or “PVP”. PVP refers to a polymer containing vinylpyrrolidone (also referred to as N-vinylpyrrolidone, N-vinyl-2-pyrrolidione and N-vinyl-2-pyrrolidinone) as a monomeric unit. Suitable vinyl-pyrrolidone polymers include poly(-vinyl-pyrrolidone) (PVP) and Plasdone® S-630, Plasdone® K-90, Plasdone® K-120.

Crosslinked polyvinylpyrrolidones are commercially available, for example, as Kollidon® CL types from BASF or as Polyplasdone® XL and Flexithix™ type PVPs from Ashland LLC.

Cross-linked polyvinylpyrrolidone is mainly used in relatively crude form as a tablet disintegrant (Kollidon CL in the range of 120 microns and Polyplasdone XL in the range of 130 microns), wherein the average particle size is greater than 100 microns.

In another embodiment, the present application involves a swellable, crosslinked PVP polymer that can be prepared directly in the form of a fine, white powder by precipitation polymerization of vinylpyrrolidone in the presence of a predetermined amount of a crosslinking agent and a free radical polymerization initiator in an organic solvent, preferably an aliphatic hydrocarbon, e.g., a C₃—C₁₀ saturated, branched or unbranched, cyclic or acyclic aliphatic hydrocarbon, and most preferably, cyclohexane or heptane, or mixtures thereof.

In various embodiments of this application, the crosslinked polymer of vinylpyrrolidone (including copolymers of vinylpyrrolidone and other monomeric materials) in a porous granular or bead form even when wetted and swollen is produced by a process wherein the monomeric material is polymerized with a controlled amount of crosslinking agent in an aqueous solution of electrolyte. The insoluble polymer is formed, and excess monomer is maintained in suspension by mechanical agitation.

In various embodiments of this invention, the crosslinked vinylpyrrolidone polymer maintains particulate form even when swollen with liquid and comprises a porous granular or bead form of polymer of vinylpyrrolidone and one or more copolymerizable monomers. For preparing a shape-maintaining, porous, granular or bead form of a vinylpyrrolidone polymer, a suspension of the monomeric material and at least a critical amount of a crosslinking agent in an aqueous solution of an electrolyte is maintained during free radical polymerization by mechanical means.

In an embodiment, the present application employs a strongly swellable, moderately crosslinked PVP polymer directly as a fine powder obtained by precipitation polymerization of vinyl pyrrolidone in the presence of a predetermined amount of a multifunctional crosslinking agent and a free radical initiator in an organic solvent.

In another embodiment, the present application employs a strongly swellable, moderately crosslinked PVP polymer based fine powder characterized by an aqueous gel volume of about 15-150 ml/g of polymer and a Brookfield viscosity in 5% aqueous solution of at least 1000-10,000 cps.

In accordance with various embodiments of this application, copolymers of methyl vinyl ether and maleic anhydride are available commercially in a range of molecular weights under the trade name “Gantrez®” (Ashland). Specifically, Gantrez® AN is a preferred copolymer. Other Gantrez® copolymers that can be used include the free acid form of the Gantrez® AN available as Gantrez® S, a mixed sodium and calcium salt of Gantrez® S available as Gantrez® MS, and half ester derivatives of Gantrez® S available as Gantrez® ES.

According to one embodiment of the present application, Stabileze QM, a cross-linked methylvinylether/maleic anhydride co-polymer, is employed. 1, 9-decadiene crosslinked copolymer, e.g., Stabileze QM-PVM/MA copolymer commercially available from Ashland LLC, is preferred. The amount of crosslinking agent generally varies from about 1 to about 5 mole percent based on the monovinyl alkyl ether. Examples of suitable crosslinking agents include the divinyl ethers of an aliphatic diol, e.g., the divinyl ethers of 1,2-ethanediol; 1,3-propanediol; 1,4-butanediol, 1,5 -pentanediol; 1,6-hexanediol; 1,7heptanediol; 1 ,8-octanediol; 1,9-nonanediol; 1,10-decanediol; 1,11-unidecanediol; and 1,12-dodecanediol, as well as the divinyl ethers of diethylene glycol, triethylene glycol, tetraethylene glycol, pentaethylene glycol; hexaethylene glycol, heptaethylene glycol, octaethylene glycol, nonaethylene glycol, decaethylene glycol and further, polyalkylene glycols up to a molecular weight of about 5900. Other suitable cross linking agents include 1,7-octadiene, 1,9-decadiene, divinylbenzene, N,N¹-bis-methylene acrylamide, acrylates such as polyethylene glycol diacrylate, trimethylolpropane triacrylate, propylene glycol diacrylate, polyhydric alcohols esterified once or twice with acrylic acid triallylamine, tetraallylehylenediamine, diallyl phthalate, and the like.

The present application comprises an orally acceptable carrier or vehicle that preferably comprises hydrophobic (oil) or hydrophilic (non-oil) components.

The hydrophobic oil or hydrophilic non-oil components of the present application can be selected from the group consisting of liquid petrolatum, petrolatum, mineral oil, glycerin, natural and synthetic oils, fats, silicone and silicone derivatives, polyvinylacetate, polyethylene glycol, propylene glycol, polypropylene glycol, poly(ethylene oxide-propylene oxide) copolymer, diethylene glycol, triethylene glycol, sorbitol, water, orally acceptable surfactant and mixtures thereof, natural and synthetic waxes such as animal waxes like beeswax, lanolin and shellac, hydrocarbons, hydrocarbon derivatives, vegetable oil waxes such as carnauba, candela and bayberry wax, vegetable oils such as caprylic/capric triglyceirdes, vegetable oils such as corn, sunflower, soy bean, castor, palm, coconut, olive, and rapeseed oil or mixtures thereof, and animal oil such as fish oil and oleic acid, and mixtures thereof.

In another embodiment, the present application discloses making mucoadhesive oral composition and of using such formulations for sustained release of active ingredients selected from the group including but not limited to an antibacterial, an anti-inflammatory, a pain reliving agent, an antioxidant, an enzyme, a flavor, a cooling agent and a sweetener.

The antibacterial compounds of the present application can be selected from the group consisting of halogenated diphenyl ether (e.g. triclosan), herbal extracts and essential oils (e.g., rosemary extract, tea extract, magnolia extract, thymol, menthol, eucalyptol, geraniol, carvacrol , citral, hinokitol, catechol, methyl salicylate, epigallocatechin gallate, epigallocatechin, gallic acid, miswak extract, sea-buckthorn extract), biguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, Zinc Chloride, Zinc Lactate, Zinc Sulfate, stannous salts, copper salts, iron salts), sanguinarine, propolis a stannous fluoride, sodium fluoride, potassium fluoride, sodium monofluorophosphate, sodium fluorosilicate, ammonium fluorosilicate, amine fluoride and ammonium fluoride. The anti-inflammatory compounds of the present application can be selected from the group including but not limited to triamcinolone (trade name: Kenalog), fluocinonide (trade name: Vanos), dexamethasone (trade name: decadron), herpes, amlexanox (aphthasol), ketorolac, flurbiprofen, ibuprofen, naproxen, indomethacin, aspirin, ketoprofen, piroxicam and meclofenamic acid.

The pain reliving compounds of the present application can be selected from the group including but not limited to benzocaine, lidocaine, procaine, prilocaine, mepivacaine, aspirin, ibuprofen, diclofenac and methyl salicylate.

The anti-oxidant compounds of the present application can be selected from the group including but not limited to vitamin E, ascorbic acid, uric acid, carotenoids, vitamin A, flavonoids, polyphenols, herbal antioxidants, melatonin, amino-indoles, lipoic acids, caffeic acid, beta-carotene, ellagic acid, epicatechin, epicatechin gallate, ferulic add, genistein, kojic acid, alpha-lipoic acid, lycopene, resveratrol, resorcinol, rosmarinic acid, silibinin, theaflavin, tocopherols, tocotrienols, trolox and ubiquinone-10.

The enzymes of the present application can be selected from the group including but not limited to Proteases; papain, bromelain, chymotrypsin, ficin and alcalase; Carbohydrases: glucoamylase, alpha-amylase, beta-amylase, dextranase and mutanas; Lipases: plant lipase, gastric lipase and pancreatic lipase and Glucoamylase, a Saccharifying glucoamylase of Aspergillus niger origin.

The flavoring agents of the present application can be selected from the group including but not limited to essential oils and various flavoring aldehydes, esters, alcohols. Examples of the essential oils include oils of spearmint, peppermint, wintergreen, sassafras, clove, sage, eucalyptus, marjoram, cinnamon, carvone, and anethole lemon, lime, grapefruit, and orange, wild oregano oil, tea tree leaf, wintergreen oil, coconut oil, myrrh oil and grapefruit peel oil.

The cooling agents of the present application can be selected from the group including but not limited to menthol, menthyl lactate, monomenthyl succinate, menthol ethylene glycol carbonate, menthol propylene glycol carbonate, menthone glycerol ketal, 3-(l-Menthoxy) propane-1,2-diol, (—)-isopulegol, WS-3 1[N-ethyl-p-menthane-3-carboxamide], WS-23 (2-isopropyl-N-2,3-trimethylbutyramide), and WS-5 [ethyl 3p-menthane-3-carboxamido)acetate].

The cooling agents of the present application can be selected from the group including but not limited to acesulfame K, aspartame, neotame, saccharin, sucralose, stevia, advantame cyclamate, sorbitol, xylitol and erythritol.

In some embodiments, the copolymers of alkyl vinyl ether-maleic acid/maleic anhydride or salts thereof is present in a suitable amount ranging from about 10 wt. % to about 20 wt. %, or from about 20 wt. % to about 30 wt. %, or from about 30 wt. % to about 40 wt. %, or from about 40 wt. % to about 50 wt. % or from about 50 wt. % to about 60 wt. % or from about 60 wt. % to about 75 wt. % based on the total weight of the mucoadhesive composition including denture adhesive composition of the present application.

In some embodiments, the cellulose ether is present in a suitable amount ranging from about 10 wt. % to about 20 wt. %, or from about 20 wt. %, to about 30 wt. %, or from about 30 wt. % to about 40 wt. %, or from about 40 wt. % to about 50 wt. % based on the total weight of the mucoadhesive composition including denture adhesive compos On of the present application.

In some embodiments, the crosslinked PVP is present in a suitable amount ranging from about 0.1 wt. % to about 1 wt. %, or from about 0.1 wt. % to about 5wt. %, or from about 5 wt. % to about 10 wt. % based on the total weight of the mucoadhesive composition including denture adhesive composition.

In some embodiments of the present application, the crosslinked methyl vinyl ether/maleic anhydride (e.g., Stabilize™ QM), or crosslinked methyl vinyl ether/maleic acid or salts thereof is present in a suitable ranging from about 0.1 wt. % to about 1 wt. %, or from about 0.1 wt. % to about 5 wt. %, or from about 5 wt. %, to about 10 wt. % based on the total weight of the mucoadhesive composition including denture adhesive composition.

In some embodiments, the hydrophobic oil or hydrophilic non-oil components based carriers is/are present in suitable amounts ranging from about 10 wt. % to about 20 wt. %, or from about 20 wt. % to about 30 wt. %, or from about 30 wt. % to about 40 wt. %, or from about 40 wt. % to about 50 wt. % or from about 50 wt. % to about 60 wt. % or from about 60 wt. % to about 75 wt. % based on the total weight of the mucoadhesive composition including denture adhesive composition.

In some embodiments, an antibacterial, an anti-inflammatory, a pain reliving agent, an antioxidant, an enzyme, a flavor, a cooling agent, and a sweetener is/are present in suitable amounts ranging from about 0.01 wt. % to about 0.1 wt. %, or from about 0.1 wt. % to about 1 wt. %, or from about 1 wt. % to about 10 wt. %, or from about 10 wt. % to about 20 wt. % based on the total weight of the mucoadhesive composition including denture adhesive composition.

In some embodiments, the alkyl vinyl ether-maleic acid/anhydride copolymers present in the composition of the present application have a weight average molecular weight of from about 500,000 to 3,000,000 suitable for denture adhesives are made by copolymerizing from about 50 mole percent of maleic anhydride, about 50 mole percent of a C₁₋₄ vinyl ether, in the presence of a free radical initiator, at about 50° to 150° C. with solvent or in a solvent-free process to produce a uniform fine powder having substantially no residual maleic acid copolymer or maleic anhydride copolymer. The copolymer of maleic anhydride and a C₁—C₄ alkyl vinyl ether preferably has a Brookfield viscosity between from about 50,000 to about 150,000 cps.

In some embodiments, the methyl vinyl ether-maleic acid copolymer or methyl vinyl ether-maleic anhydride copolymer (PVM/MA copolymer) present in the composition of the present application has a number average molecular weight of between about 50,000 to about 500,000 Daltons and wherein from about 50 wt. % to about 100 wt. % of the carboxyl units in the polymer are converted to a mixture of metal salts, and further, wherein, the metals are selected from the group consisting of calcium, sodium, strontium, zinc, magnesium, iron, boron, aluminum, potassium, vanadium, chromium, manganese, nickel, copper, yttrium, titanium, and mixtures thereof.

In some embodiments, the salts of a copolymer of alkyl vinyl ether-maleic acid or salts of copolymer of alkyl vinyl ether-maleic anhydride have a specific viscosity of from 2.5 to 5.0 when measured as a 1% w/v solution in methyl ethyl ketone (MEK) solution at 25° C.

As disclosed herein, in some embodiments, the methyl vinyl ether has a weight average molecular weight (Mw) ranging from about 700,000 to about 3,000,000. In some embodiments, the methyl vinyl ether copolymer has a weight average molecular weight (Mw) ranging from 500,000 to 3,000,000.

As disclosed herein, in some embodiments, the methyl vinyl ether-maleic acid copolymer or methyl vinyl ether-maleic anhydride copolymer or salts thereof (PVM/MA copolymer) have a number average molecular weight ranging from 50,000 to about 500,000.

As disclosed herein, in some embodiments the sodium carboxymethylcellulose has a molecular weight of from about 500,000 to about 1,200,000 Daltons. In some embodiments, the sodium carboxymethylcellulose has a molecular weight ranging from 600,000 to about 800,000 Daltons.

According to another embodiment of the present application, it is contemplated to employ at least one excipient. Suitable excipients for the purposes of this application are selected from the group consisting of a preservative, a flavoring agent, a colorant, a sweetener, a plasticizer, a binder, a thickener, vehicles, colorant, carrier, flavor, fragrance, sensate, arid mixtures thereof.

In one embodiment, the mucoadhesive composition is a an oral gel, an oral ointments, an oral lotion, buccal composition, a sublingual composition, a palatal composition, and a denture adhesive composition in the form of a dentifrice, denture cleanser, chewing gum, lozenge, mouth spray, mousse, foam, dental implement (dental floss or dental tape), dental solution, denture cleanser, toothpaste, tooth powder, topical oral gel, mouth rinse, mouth spray, denture product, dissolvable film, strip, oral tablet, aerosol, wafer, chewing gum or breath freshener.

In one embodiment, it is contemplated to use a material that is suitable for mucoadhesive composition including denture adhesive, particularly, it is safe and palatable at relevant concentrations tor use in a hygiene composition, such as liquid, powder, cream, gel, thermoplastic solid, hydrogel or combinations thereof.

The following examples are presented for purposes of demonstrating, but not limiting, the preparation and use of the polymers. In the examples, the following abbreviations are used:

PVP: Polyvinylpyrrolidone

MVE: Methyl vinyl ether

MA: Maleic anhydride/Maleic acid

CMC: Carboxymethyl cellulose

wt %: Weight percent

Flexithix: Lightly to moderately crosslinked polyvinyl pyrrolidone

Polyplasdone XL-10: Self-crosslinked polyvinyl pyrrolidone

Stabileze QM: Poly(methylvinylether/maleic anhydride) crosslinked with 1,9-decadiene

PVP K-90: Polyvinyl pyrrolidone powder average molecular weight of 1,300,000 in Daltons

Gantrez MS-955: Sodium/Calcium mixed salt of Poly(methylvinylether/maleic anhydride)

Aqualon CMC 7H3SXF: Sodium Carboxymethyl cellulose

Germaben II : Liquid contains propylene glycol, propylparaben, methylparaben, and diazolidinyl urea.

PVP K-15: Polyvinylpyrrolidone powder average molecular weight of 8,000 in Daltons

Further, certain aspects of the present application are illustrated in detail by way of the following examples. The examples are given herein for illustration of the application and are not intended to be limiting thereof.

### EXAMPLES

Performance enhancer materials that are useful for this invention are hydrophilic polymers that are swellable air partially soluble in water/saliva. These polymers can be characterized by the pronounced affinity of their chemical structures for aqueous solutions in which they swell rather than dissolve. These polymers range from being low to mildly absorbing, typically retaining 1-30 wt. % of water within their structure. Super absorbing polymers that can retain water above 30% wt. of their weight cannot be useful for this application as they tend to destabilize the denture adhesive cream by absorbing excess saliva promoting oozing.

TABLE 1
Performance enhancers and adhesive polymers

| | Molecular | Dose | Actual |
|---|---|---|---|
| Chemical description | weight | range | dosage |
| Lightly crosslinked polyvinyl | N/A | 0.1-10% | 2.5-5% |
| pyrrolidone (Flexithix) | | | |
| Self-crosslinked polyvinyl | N/A | 0.1-10% | 2.5-5% |
| pyrrolidone (Polyplasdone XL-10) | | | |
| Poly (methylvinylether/maleic | N/A | 0.1-10% | 2.5-5% |
| anhydride) crosslinked with 1,9- | | | |
| decadiene (Slabileze QM) | | | |
| Polyvinyl pyrrolidone (PVP- K90) | 1,300,000 | 0.1-10% | 2.5-5% |
| Sodium/Calcium mixed salt of Poly | 1,200,000 | 10-60% | 25-35% |
| (methylvinylether/maleic | | | |
| anhydride)- (Gantrez MS-955) | | | |
| Sodium Carboxymethyl cellulose | 700,000 | 10-60% | 20-30% |
| (Aqualon CMC 7H3SXF) | | | |

### Comparative Example 1: Standard Oil-Based Denture Adhesive formulation)

200 g batch of standard oil-based denture adhesive formulation was prepared in a KitchenAid planetary mixer equipped with a digitally controlled constant temperature heated jacket. The heating jacket was set to maintain a constant temperature of 194° F. (90° C.). Components required to make denture adhesive cream were added as per Table 1. Petrolatum (40 g) and white mineral oil (40 g) were charged into the heated bowl of the KitchenAid mixer and mixed at low speed for 10 minutes till petrolatum completely melted and produced a homogeneous transparent liquid. Sodium Carboxymethyl cellulose (54 g) was charged into the KitchenAid and mixed vigorously with the oils for 30 minutes till uniformly dispersed. Mixed Ca/Na salt of Poly (MVE/MA) (66 g) was then charged into the KitchenAid and mixed at medium speed for another 30 minutes to yield a uniform consistency paste. Suitable color was added and mixed till uniformly dispersed. The heat was turned off and the denture adhesive cream was allowed to cool down to room temperature over a period of 2 hours under continuous medium speed stirring. After cooling down to room temperature, 50 g denture adhesive sample was packed in proper dispensing tube and heat sealed. Remaining denture adhesive was packed in clear glass jars and used for storage stability studies.

### Example 2: Experimental Oil-Based Denture Adhesive formulation (E12) 5% PVP K-90

200 g batch of experimental oil-based denture adhesive formulation was prepared in a KitchenAid planetary mixer equipped with a digitally controlled constant temperature heated jacket. The heating jacket was set to maintain a constant temperature of 194° F. (90° C.). Components required to make denture adhesive cream were added as per Table 1. Petrolatum (40 g) and white mineral oil (40 g) were charged into the heated bowl of the KitchenAid mixer and mixed at low speed for 10 minutes till petrolatum completely melted and produced a homogeneous transparent liquid, Non-crosslinked PVP (PVP-K-90) (10 g) was charged into the KitchenAid and mixed vigorously with the oils for 15 minutes till uniformly dispersed. Sodium Carboxymethyl cellulose (50 g) was charged into the KitchenAid and mixed vigorously with the oils for 30 minutes till uniformly dispersed. Mixed Ca/Na salt of Poly (MVE/MA) (60 g) was then charged into the KitchenAid and mixed at medium speed for another 30 minutes to yield a uniform consistency paste. Suitable color was added and mixed till uniformly dispersed. The heat was turned off and the denture adhesive cream was allowed to cool down to room temperature over a period of 2 hours under continuous medium speed stirring. After cooling down to room temperature, 50 g denture adhesive sample was packed in proper dispensing tube and heat sealed. Remaining denture adhesive was packed in clear glass jars and used for storage stability studies.

### Example 3: Experimental Oil-based Denture Adhesive formulation (E13) 13 Stabileze QM

200 g batch of experimental oil-based denture adhesive formulation was prepared in a KitchenAid planetary mixer equipped with a digitally controlled constant temperature heated jacket. The heating jacket was set to maintain a constant temperature of 194° F. (90° C.). Components required to make denture adhesive cream were added as per Table 1. Petrolatum (40 g) and white mineral oil (40 g) were charged into the heated howl of the KitchenAid mixer and mixed at low speed for 10 minutes till petrolatum completely melted and produced a homogeneous transparent liquid. Crosslinked Poly (MVE/MA) (6 g) was charged into the KitchenAid and mixed vigorously with the oils for 15 minutes till uniformly dispersed. Sodium Carboxymethyl cellulose (50 g) was charged into the KitchenAid and mixed vigorously with the oils for 30 minutes till uniformly dispersed. Mixed Ca/Na salt of Poly (MVE/MA) (64 g) was then charged into the KitchenAid and mixed at medium speed for another 30 minutes to yield a uniform consistency paste. Suitable color was added and mixed till uniformly dispersed. The heat was turned off and the denture adhesive cream was allowed to cool down to room temperature over a period of 2 hours under continuous medium speed stirring. After cooling down to room temperature, 50 g denture adhesive sample was packed in proper dispensing tube and heat sealed. Remaining denture adhesive was packed in clear glass jars and used for storage stability studies.

### Example 4: Experimental Oil-Based Denture Adhesive Formulation (E14) 3% Polyplasdone XL-10

200 g batch of experimental oil-based denture adhesive formulation was prepared in a KitchenAid planetary mixer equipped with a digitally controlled constant temperature heated jacket. The heating jacket was set to maintain a constant temperature of 194° F. (90° C.). Components required to make denture adhesive cream were added as per Table 1, Petrolatum (40 g) and white mineral oil (40 g) were charged into the heated bowl of the KitchenAid mixer and mixed at low speed for 10 minutes till petrolatum completely melted and produced a homogeneous transparent liquid. Crosslinked PVP Polyplasdone XL-10 (6 g) was charged into the KitchenAid and mixed vigorously with the oils for 15 minutes till uniformly dispersed. Sodium Carboxymethyl cellulose (50 g) was charged into the KitchenAid and mixed vigorously with the oils for 30 minutes till uniformly dispersed. Mixed Ca Na salt of Poly (MVE/MA) (64 g) was then charged into the KitchenAid and mixed at medium speed for another 30 minutes to yield a uniform consistency paste. Suitable color was added and mixed till uniformly dispersed. The heat was turned off and the denture adhesive cream was allowed to cool down to room temperature over a period of 2 hours under continuous medium speed stirring. After cooling down to room temperature, 50 g denture adhesive sample was packed in proper dispensing tube and heat sealed. Remaining denture adhesive was packed in clear glass jars and used for storage stability studies.

### Example 5: Experimental Oil-Based Denture Adhesive Formulation (E15) 3% Flexithix

200 g batch of experimental oil-based denture adhesive formulation was prepared in a KitchenAid planetary mixer equipped with a digitally controlled constant temperature heated jacket. The heating jacket was set to maintain a constant temperature of 194° F. (90° C.). Components required to make denture adhesive cream were added as per Table 1. Petrolatum (40 g) and white mineral oil (40 g) were charged into the heated bowl of the KitchenAid mixer and mixed at low speed for 10 minutes till petrolatum completely melted and produced a homogeneous transparent liquid. Crosslinked PVP, Flexithix (6 g) was charged into the KitchenAid and mixed vigorously with the oils for 15 minutes till uniformly dispersed. Sodium Carboxymethyl cellulose (50 g) was charged into the KitchenAid and mixed vigorously with the oils for 30 minutes till uniformly dispersed. Mixed Ca/Na salt of Poly (MVE/MA) (64 g) was then charged into the KitchenAid and mixed at medium speed for another 30 minutes to yield a uniform consistency paste. Suitable color was added and mixed till uniformly dispersed. The heat was turned off and the denture adhesive cream was allowed to cool down to room temperature over a period of 2 hours under continuous medium speed stirring. After cooling down to room temperature, 50 g denture adhesive sample was packed in proper dispensing tube and heat sealed. Remaining denture adhesive was packed in clear glass jars and used for storage stability studies.

### Example 6: Experimental Oil-Based Denture Adhesive Formulation (E16) 2.5% Polyplasdone XL-10+2.5% Flexithix

200 g batch of experimental oil-based denture adhesive formulation was prepared in a KitchenAid planetary mixer equipped with a digitally controlled constant temperature heated jacket. The heating jacket was set to maintain a constant temperature of 194° F. (90° C.). Components required to make denture adhesive cream were added as per Table 1. Petrolatum (40 g) and white mineral oil (40 g) were charged into the heated bowl of the KitchenAid mixer and mixed at low speed for 10 minutes till petrolatum completely melted and produced a homogeneous transparent liquid. Crosslinked Polyplasdone XL-10 (5 g) and Flexithix (5 g) was charged into the KitchenAid and mixed vigorously with the oils for 15 minutes till uniformly dispersed. Sodium Carboxymethyl cellulose (50 g) was charged into the KitchenAid and mixed vigorously with the oils for 30 minutes till uniformly dispersed. Mixed Ca/Na salt of Poly (MVE/MA) (60 g) was then charged into the KitchenAid and mixed at medium speed for another 30 minutes to yield a uniform consistency paste. Suitable color was added and mixed till uniformly dispersed. The heat was turned off and the denture adhesive cream was allowed to cool down to room temperature over a period of 2 hours under continuous medium speed stirring. After cooling down to room temperature, 50 g denture adhesive sample was packed in proper dispensing tube and beat sealed. Remaining denture adhesive was packed in clear glass jars and used for storage stability studies.

### Example 7: Experimental Oil-Free Denture Adhesive Formulation (OF1) 1% PVP K-15

200 g batch of standard oil-free denture adhesive formulation was prepared in a KitchenAid planetary mixer equipped with a digitally controlled constant temperature heated jacket. The heating jacket was set to maintain a constant temperature of 115° F. (˜46° C.). Components required to make denture adhesive cream were added as per Table 2. Propylene glycol (38.5 g) and PEG-400 (38.5 g) were charged into the heated bowl of the KitchenAid mixer and mixed at low speed for 20 minutes till homogeneous transparent liquid was obtained. PVP K-15 (2 g) was added to the KitchenAid mixer and mixed at high speed for 45 minutes till a clear solution was obtained. Sodium Carboxymethyl cellulose (54 g) was then charged into the KitchenAid and mixed vigorously for 30 minutes till uniformly dispersed. Mixed Ca/Na salt of Poly (MVE/MA) (66 g) was then charged into the KitchenAid and mixed at medium speed for another 30 minutes to yield a uniform consistency paste. Germaben II preservative (1 g) was added after that and mixed for additional 15 minutes. The heat was turned off and the denture adhesive cream was allowed to cool down to room temperature over a period of 2 hours under continuous medium speed stirring. At cooling down to room temperature, 50 g denture adhesive sample was packed in proper dispensing tube and heat sealed. Remaining denture adhesive was packed in clear glass jars and used for storage stability studies.

### Example 8: Experimental Oil-Free Denture Adhesive Formulation (OF2) 1% Flexithix

200 g batch of standard oil-free denture, adhesive formulation was prepared in a KitchenAid planetary mixer equipped with a digitally controlled constant temperature heated jacket. The heating jacket was set to maintain a constant temperature of 115° F. (˜46° C.). Components required to make denture adhesive cream were added as per Table 2. Propylene glycol (39.5 g) and PEG-400 (39.5 g) were charged into the heated bowl of the KitchenAid mixer and mixed at low speed for 20 minutes till homogeneous transparent liquid was obtained. Crosslinked PVP, Flexithix (2 g was added to the KitchenAid mixer and mixed at high speed for 45 minutes till a clear thin gel was obtained. Sodium Carboxymethyl cellulose (54 g) was then charged into the KitchenAid and mixed vigorously for 30 minutes till uniformly dispersed. Mixed Ca/Na salt of Poly (MVE/MA) (66 g) was then charged into the KitchenAid and mixed at medium speed for another 30 minutes to yield a uniform consistency paste. Germaben II preservative (1 g) was added after that and mixed for additional 15 minutes. The heat was turned off and the denture adhesive cream was allowed to cool down to room temperature over a period of 2 hours under continuous medium speed stirring. After cooling down to room temperature, 50 g denture adhesive sample was packed in proper dispensing tube and heat sealed. Remaining denture adhesive was packed in clear glass jars and used for storage stability studies.

TABLE 2
Oil-based Denture adhesive compositions

| Components | E1 | E12 | E13 | E14 | E15 | E16 |
|---|---|---|---|---|---|---|
| Petrolatum (g) | 40.0 | 40.0 | 40.0 | 40.0 | 40.0 | 40.0 |
| Mineral oil (g) | 40.0 | 40.0 | 40.0 | 40.0 | 40.0 | 40.0 |
| PVP K-90 (g) | 0.0 | 10.0 | 0.0 | 0.0 | 0.0 | 0.0 |
| Crosslinked Poly | 0.0 | 0.0 | 6.0 | 0.0 | 0.0 | 0.0 |
| (MVE/MA) (g) | | | | | | |
| Flexithix (g) | 0.0 | 0.0 | 0.0 | 0.0 | 6.0 | 5.0 |
| Polyplasdone XL-10 (g) | 0.0 | 0.0 | 0.0 | 6.0 | 0.0 | 5.0 |
| Mixed Ca/Na salt of | 66.0 | 60.0 | 64.0 | 64.0 | 64.0 | 60.0 |
| Poly (MVE/MA (g)) | | | | | | |
| Sodium Carboxymethyl | 54.0 | 50.0 | 50.0 | 50.0 | 50.0 | 50.0 |
| cellulose (g) | | | | | | |
| Total (g) | 200.0 | 200.0 | 200.0 | 200.0 | 200.0 | 200.0 |

### Example 9: Denture Adhesive Test Method

Adhesion forces and relative denture adhesive film thickness were recorded on TA.XT Plus texture analyzer instruments from Stable Microsystems Texture Technologies Corp., equipped with a 50 Kg load cell and interfaced with a PC running Exponent software version 6.1.11.0. The texture analyzer instruments were equipped with a custom-built denture shaped plexiglass probe-fixture assembly. Artificial saliva infusion between the walls of the plexiglass probe-fixture assembly was achieved using peristaltic pumps. Real-time time-lapse images of denture adhesive creams undergoing adhesion performance evaluation on the texture analyzer instruments were recorded using a Canon EOS 5d Mark IV Digital SLR camera.

Sample tubes containing denture adhesive creams were vented for trapped air and with the cap on, squished several times to homogenize the contents inside the tube. The first 100 mg of material coming out of the tube was always discarded.

Three nearly identical strips of denture adhesive cream were weighed out into the denture shaped cavity of the bottom plexiglass fixture on an analytical balance. Total mass of denture adhesive cream applied to the bottom fixture for each test was 2.0 (+0.1) g. The bottom fixture was mounted on to the texture analyzer instrument and the top probe was moved down into the cavity of the bottom fixture to uniformly spread the denture adhesive cream and fill the bottom half of the cavity under a precise compression force of 4.5 Kg. Infusion of artificial saliva was initiated. For the entire duration of the experiment, the denture adhesive cream remained in contact and submerged under a thin level of artificial saliva which was continuously refreshed at a flow rate of 1 ml/minute. The 7-hour test sequence was initiated once the denture adhesive cream layer was fully covered with artificial saliva. Adhesion force (in Newtons) at instances of mastication and thickness of denture adhesive film between the top and the bottom probe (in mm) were continuously measured for the duration of the experiment. Each sample of prepared denture adhesive formulations was subjected to four consecutive experimental runs. The average plots from 4 runs for each sample were compared for adhesion performance, and thickness variation. Total adhesion for each sample was calculated as the area under the curve for the average plot of the 4 consecutive runs for each sample.

TABLE 3
Oil-free Denture adhesive compositions

| Components (g) | OF1 | OF2 |
|---|---|---|
| Propylene glycol | 38.5 | 38.5 |
| PEG-400 | 38.5 | 38.5 |
| PVP K-15 | 2.0 | 0.0 |
| Flexithix | 0.0 | 2.0 |
| Mixed Ca/Na salt of Poly (MVE/MA) | 66.0 | 64.0 |
| Sodium Carboxymethyl cellulose | 54.0 | 50.0 |
| Germaben II | 1.0 | 1.0 |
| Total (g) | 200.0 | 200.0 |

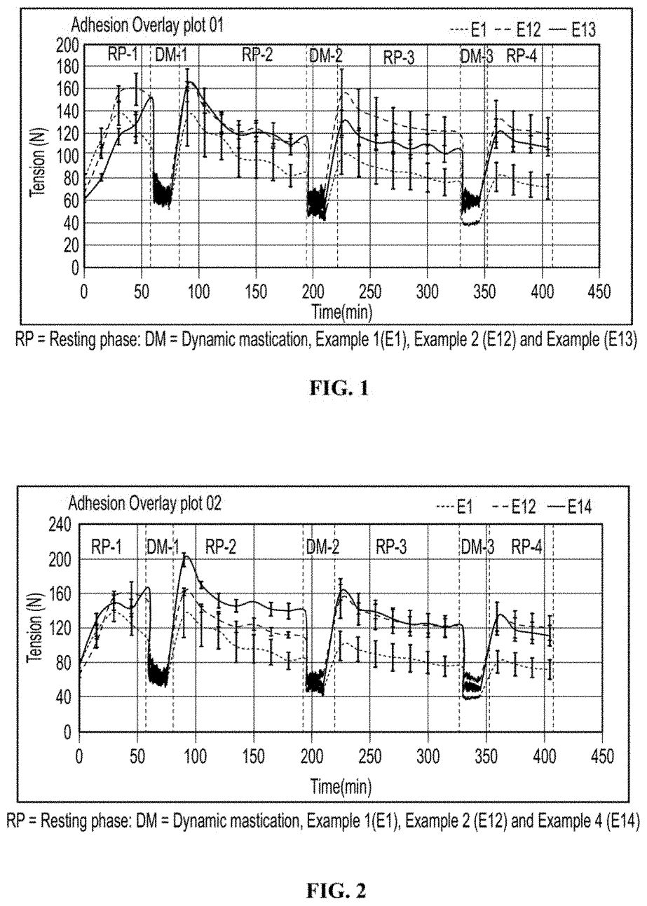

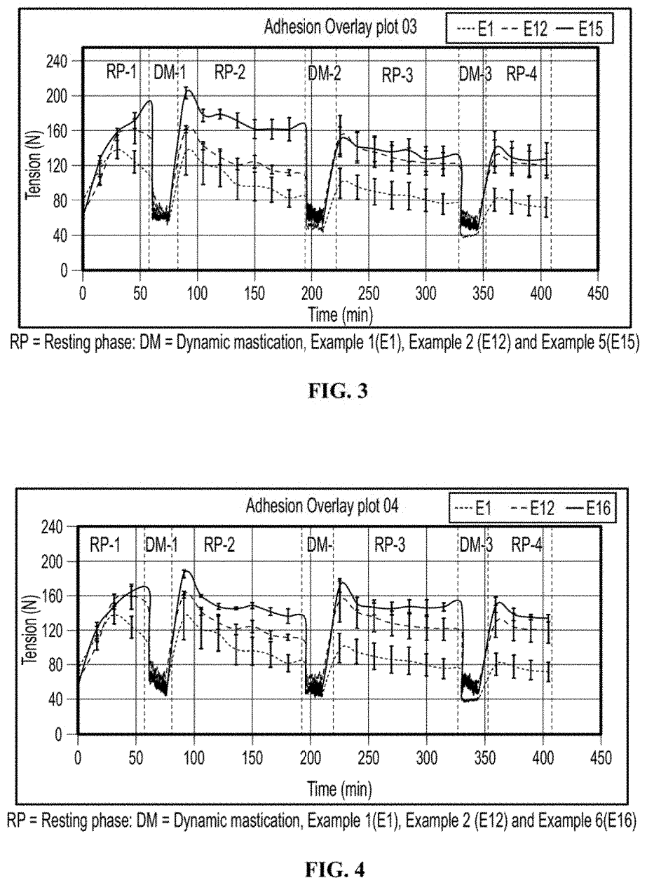

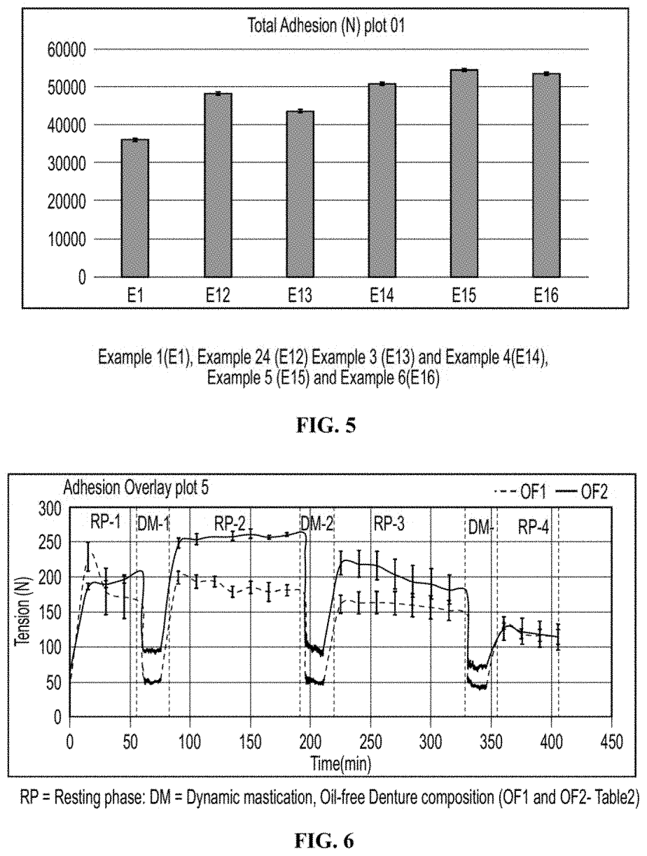

FIGS. 1-4 represent overlay of average adhesion profiles of sample oil-based denture adhesives E1, E12-16. Adhesion profile trace of each denture adhesive sample is an average of four consecutive experimental runs. Each overlay plot is demarcated into alternating phases of slow chewing motion or Resting Phase (RP) and rapid chewing motion or Dynamic Mastication (DM). Adhesion forces are recorded and reported in Newton (N). FIG. 5 shows the Total Adhesion for each representative denture adhesive formulations. Total Adhesion (in N) is measured as the total area under the curve for the average adhesion profile trace for each representative denture adhesive formulations.

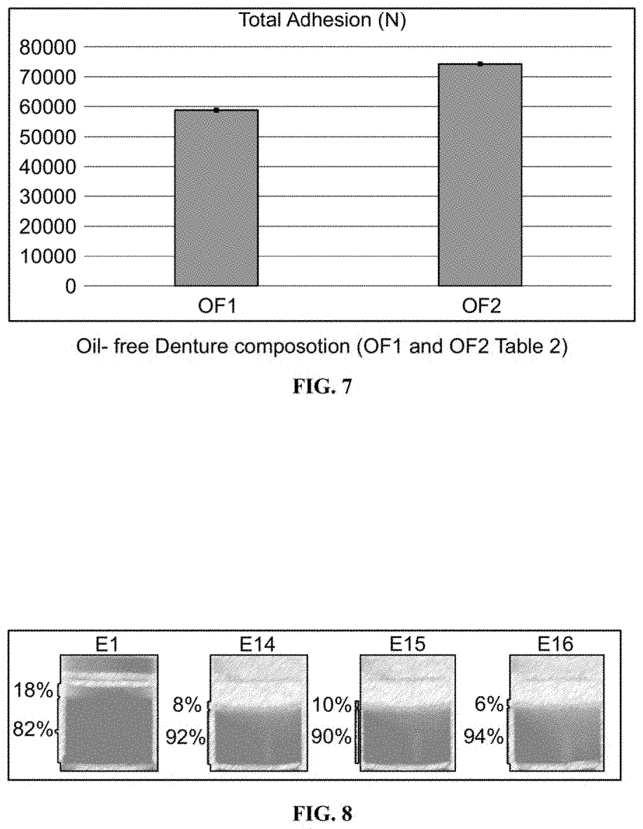

FIGS. 6 represents overlay of average adhesion profiles of sample oil-free denture adhesives OF1 and OF2. Adhesion profile trace of each denture adhesive sample is an average of four consecutive experimental runs. Each overlay plot is demarcated into alternating phases of slow chewing motion or Resting Phase (RP) and rapid chewing motion or Dynamic Mastication (DM). Adhesion forces are recorded and reported in Newton (N). FIG. 7 shows the Total Adhesion for each representative oil-free denture adhesive formulations. Total Adhesion (in N) is measured as the total area under the curve for the average adhesion profile trace for each representative denture adhesive formulations.

### Example 11: Stability of Denture Adhesive Formulations

FIG. 8 shows stability of selected oil-based denture adhesive formulations upon storage at 45° C. for 12 weeks. Degree of syneresis of hydrophobic carrier in the formulation seen as oil separation upon storage at 45° C. for 12 weeks was calculated as % height of formulation that separated out as oil due to syneresis and considered as the measure of stability of the formulations. Lowed the oil separation %, better was the stability of the sample after 12 weeks of storage at 45° C.

### Example 12: Mucoadhesive Formulations

Examples of the orally acceptable mucoadhesive formulations are described in tables 4 and 5.

TABLE 4
Oil-based Mucoadhesive compositions

| Components | E17 | E18 | E19 | E20 | E21 |
|---|---|---|---|---|---|
| Petrolatum (g) | 35.0 | 35.0 | 35.0 | 35.0 | 35.0 |
| Mineral oil (g) | 35.0 | 35.0 | 35.0 | 35.0 | 35.0 |
| Benzocaine (g) | 10 | 10.0 | 10.0 | 10.0 | 10.0 |
| Crosslinked Poly | 0.0 | 0.0 | 6.0 | 0.0 | 0.0 |
| (MVE/MA) (g) | | | | | |
| Flexithix (g) | 0.0 | 6.0 | 0.0 | 0.0 | 5.0 |
| Polyplasdone XL-10 (g) | 0.0 | 0.0 | 0.0 | 6.0 | 5.0 |
| Mixed Ca/Na salt of | 66.0 | 64.0 | 64.0 | 64.0 | 60.0 |
| Poly (MVE/MA (g) | | | | | |
| Sodium Carboxymethyl | 54.0 | 50.0 | 50.0 | 50.0 | 50.0 |
| cellulose (g) | | | | | |
| Total (g) | 200.0 | 200.0 | 200.0 | 200.0 | 200.0 |

TABLE 5
Oil-free mucoadhesive compositions

| Components (g) | OF3 |
|---|---|
| Propylene glycol | 3.5 |
| PEG-400 | 3.5 |
| PVP K-15 | 2.0 |
| Benzocaine | 8.0 |
| Flexithix | 2.0 |
| Mixed Ca/Na salt of Poly (MVE/MA) | 66.0 |
| Sodium Carboxymethyl cellulose | 54.0 |
| Germaben II | 1.0 |
| Total (g) | 200.0 |

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing, from the concept, spirit and scope of the disclosed and/or claimed inventive concept(s). All such similar substitutes and modifications

What is claimed is:

1. A mucoadhesive composition comprising:
i. about 10 to about 75 wt. % of a maleic acid or maleic anhydride copolymer or salts thereof;
ii. about 10 to about 50 wt. % of at least one cellulose ether;
iii. about 0.1 to about 10 wt. % of a crosslinked polyvinyl pyrrolidone polymer that is swellable, but not soluble in water; and
iv. about 30 to about 70 wt. % of an orally acceptable carrier.

2. The mucoadhesive composition according to claim 1, wherein the maleic acid or maleic anhydride copolymer comprises repeating units of maleic acid or maleic anhydride or salts thereof.

3. The mucoadhesive composition according to claim 1, wherein the maleic acid or maleic anhydride copolymer comprises maleic acid or maleic anhydride and a C₁—C₄ alkyl vinyl ether, having a predetermined weight average molecular weight of about 500,000 to 3,000,000 made by copolymerizing about 50 mole percent of maleic acid or maleic anhydride, about 50 mole percent of a C₁₋₄ alkyl vinyl ether, in the presence of a free radical initiator, at about 50 to 150° C. in a solvent or solvent free process to produce a uniform, fine powder having substantially no residual maleic anhydride.

4. The mucoadhesive composition according to claim 3, wherein the methyl vinyl ether-maleic acid copolymer or methyl vinyl ether-maleic anhydride copolymer has a number average molecular weight of between about 50.000 and about 500,000 wherein about 50 to about 100 wt. % of the carboxyl units in the polymer are converted to a mixture of metal salts selected from the group consisting of calcium, sodium, strontium, zinc, magnesium, iron, boron, aluminum, potassium, vanadium, chromium, manganese, nickel, copper, yttrium, titanium, and mixtures thereof.

5. The mucoadhesive composition according to claim 4, wherein the salts of the copolymer of methyl vinyl ether-maleic acid or methyl vinyl ether-maleic anhydride has a specific viscosity of from about 2.5 to 5.0 when measured as a 1% w/v solution in methyl ethyl ketone (MEK) solution at 25° C.

6. The mucoadhesive composition according to claim 5, wherein the copolymer of methyl vinyl ether-maleic acid or methyl vinyl ether-maleic anhydride or salts thereof comprises from about 18 to about 66 wt. % of said composition.

7. The mucoadhesive composition according to claim 6, wherein the methyl vinyl ether-maleic acid or methyl vinyl ether-anhydride copolymer has a weight average molecular weight of from about 700,000 to about 3,000,000.

8. The mucoadhesive composition according to claim 1, wherein the cellulose ether is selected from the group consisting of methylcellulose, hydroxyethylcellulose, hydroxypropylcellulose, hydroxypropylmethylcellulose, sodium carboxymethylcellulose and mixtures thereof.

9. The mucoadhesive composition according to claim 8, wherein the cellulose ether is sodium carboxymethylcellulose (NaCMC).

10. The mucoadhesive composition according to claim 9, wherein the carboxymethylcellulose has a number average molecular weight range of from 500,000 to 1,200,000 Daltons.

11. The mucoadhesive composition according to claim 10, wherein the sodium carboxymethylcellulose has a number average molecular weight range of from about 600,000 to about 800,000 Daltons.

12. The mucoadhesive composition according claim 11, wherein the sodium carboxymethyl cellulose comprises from about 15 to about 50 wt. % of the mucoadhesive composition.

13. The mucoadhesive composition according to claim 1, wherein the crosslinked polyvinyl pyrrolidone is in the form of a fine, free flowing white powder.

14. The mucoadhesive composition according to claim 1, further comprising about 0.001 to about 5 wt. % of an excipient selected from the group consisting of a preservative, a colorant, a sweetener, a pigment, a plasticizer, a binder, a thickener, a vehicle, a butler, a humectant, a surfactant, a detergent, a foaming agent, a gelling agent, a stabilizer, an abrasive, an antioxidant carrier, a desensitizing agent, an fragrance, a sensate and mixtures thereof.

15. The mucoadhesive composition according to claim 1, further comprising about 0.01 to about 20 wt. % of an active ingredient selected from the group consisting of an antibacterial, an anti-inflammatory, a pain reliving agent, an antioxidant, an enzyme, a flavor, a cooling agent, a sweetener and mixtures thereof.

16. The mucoadhesive composition according to claim 1, wherein said orally acceptable carrier includes hydrophobic oil or hydrophilic non-oil components selected from the group consisting of petrolatum, mineral oil, olive oil, vegetable oil, silicon or glycerin, polyethylene glycol, propylene glycol, poly(ethylene oxide-propylene oxide) copolymer, diethylene glycol, triethylene glycol, sorbitol, water, orally acceptable surfactants and mixtures thereof.

17. The mucoadhesive composition according to claim 1, wherein said composition is an oral gel, an oral ointments, an oral lotion, a buccal composition, a sublingual composition, a palatal composition, and a denture adhesive composition in the form of a powder, cream, paste, liquid, gel. aerosol, and/or wafer.

18. A mucoadhesive oral composition comprising:
i. about 15 to about 70 wt. % of a maleic acid copolymer selected from the group consisting of methyl vinyl ether-maleic acid copolymer or methyl vinyl ether-maleic anhydride copolymer or salts thereof;
ii. about. 10 to about. 50 wt. % of at least one carboxymethylcellulose;
iii. about 0.1 to about 10 wt. % of a swellable, water insoluble crosslinked polyvinyl pyrrolidone;
iv. about 10 to about 60 wt. % of an orally acceptable carrier; and
v. about 0.01 to about 20 wt. % of an active ingredient selected from the group consisting of an antibacterial, an anti-inflammatory, a pain reliving agent, an antioxidant, an enzyme, a flavor, a cooling agent, a sweetener and mixtures thereof and further wherein the composition is either a gel or a liquid.

19. A denture adhesive composition comprising:
about 15 to about 70 wt. % of a maleic acid copolymer selected from the group consisting of methyl vinyl ether-maleic acid copolymer or methyl vinyl ether-maleic anhydride copolymer or salts thereof,
ii. about 10 to about 50 wt. % of at least one carboxymethylcellulose;
iii. about 0.1 to about 10 wt. % of a swellable, water insoluble crosslinked polyvinyl pyrrolidone;
iv. about 10 to about 60 wt. % of an orally acceptable carrier; and
vi. about 0.01 to about 20 wt. % of an active ingredient selected from the group consisting of an antibacterial, an anti-inflammatory, a pain reliving agent, an antioxidant, an enzyme, a flavor, a cooling agent, a sweetener and mixtures thereof and further wherein the composition is either a gel or a liquid.

20. The denture adhesive composition according to claim 19, wherein the composition is coated in an effective denture holding amount on the inner surface of a denture.

21. The denture adhesive composition according, to claim 19, wherein said composition is used for improving denture adhesive performance including strength and duration of adhesive hold, film thickness, cushioning effects, ooze control, shelf-life storage stability, easy cleanability, extended holding time and viscosity building properties.

22. A method of adhering a denture having a biocontact surface to a gum or a roof of a mouth, the method comprising treating the biocontact surface of the denture with a denture adhesive composition comprising:
about 15 to about 70 wt. % of a maleic acid copolymer selected from the group consisting of methyl vinyl ether-maleic acid copolymer or methyl vinyl ether-maleic anhydride copolymer or salts thereof;
ii. about 10 to about 50 wt. % of at least one carboxymethylcellulose
iii. about 0.1 to about 10 wt. % of a swellable, water insoluble crosslinked polyvinyl pyrrolidone: and
iv. about 10 to about 60 wt. % of an orally acceptable carrier; and
v. about 0.01 to about 20 wt. % of an active ingredient selected from the group consisting of an antibacterial, an anti-inflammatory, a pain reliving agent, an antioxidant, an enzyme, a flavor, a cooling agent, a sweetener and mixtures thereof.
